(19) United States
(12) Patent Application Publication
(10) Pub. No.: US 2007/0157933 A1
(43) Pub. Date: Jul. 12, 2007

(54) Clip-in nostril expander

(76) Inventor: Glen B. Reed, St. George, UT (US)

Correspondence Address: M. REID RUSSELL, 854 WEST 3390 SOUTH, HURRICANE, UT, 84737, US

(21) Appl. No.: 11/717,444

(22) Filed: Mar. 12, 2007

Related U.S. Application Data
(63) Continuation-in-part of application No. 11/273,412, filed on Nov. 15, 2005.

(51) Int. Cl. A62B 7/10; A61M 29/00

(52) U.S. Cl. 128/206.11; 606/196

(57) ABSTRACT

A clip-in nostril expander includes a pair of like short cylindrical dilators that are connected by a strap from a soft thermoplastic material, and the dilators are for insertion into each of a person's nostrils, opening each nostril to a free flow of air. The dilators attach to opposite ends of the strap, with the single unit to retain a shape or bend as is formed therein. For installation of the clip-in nostril expander, the strap is bent at a mid-point and that bend is held between a person's thumb and forefinger. The dilators are then inserted into each of the person's nostrils and the strap bend is fitted onto the person's septum, holding the dilators in the person's nostrils.

### BACKGROUND OF THE INVENTION

This application is a Continuation in Part application of application Ser. No. 11/273,412, for a “CLIP-IN NOSTRIL EXPANDER” filed Nov. 15, 2005 that is abandoned with the entry of this Continuation in Part application.

### FIELD OF THE INVENTION

The present invention pertains to devices for inserting into each nostril of a human's nose to hold open the breathing passages thereof.

### PRIOR ART

Currently, there are a number of breathing aids on the market for opening a person's nasal passages. One such device is a band aid type device for positioning across a person's nose to hold open the nostrils at their outer walls, and an example of this type of device is shown in a U.S. Pat. No's. 5,549,103 and 5,653,224to Johnson. Further, and more similar to the present invention, U.S. Pat. No. 3,710,799; to Caballero, to Rella, U.S. Pat. No. 4,105,035; to Rezakhany, U.S. Pat. No. 4,414,977; to Lee, U.S. Pat. No. 5,665,104; and to Filis, U.S. Pat. No. 6,004,342, show devices for fitting into a person's nostrils. Like the invention, the Lee U.S. Pat. No. 5,665,104 and Filis U.S. Pat. No. 6,004,342 patents both show a pair of cylinders connected by a strap, with each cylinder for fitting into a person's nostril. Where the Filis '342 patent shows the inserts as truncated cylinders that attempt to conform to the shape of a person's nasal cavities, the Lee '104 patent, like the invention, shows the cylinders as being regular cylinders. Distinct from both the Filis and Lee patents, as well as the other cited patents, the invention does not rely on the shape or diameter of the cylinders to maintain the positioning of the individual cylinders in the person's nostrils. Rather, the strap connecting the cylinders of the invention is formed to bend and retain that bend, clamping onto the surface of the septum between the person's nostrils. Which strap, to remove the invention, is easily bent out of clamping engagement, and is gripped between the person's thumb and fore finger to pull the cylinders out of the person's nostrils. Where, however, a U.S. Pat. No. 6,863,066 shows a use of a metal tube for clamping to hold loop ends of a flexible line of a nasal dilator, that tub is not meant for, and clearly is not suitable for, use for clamping to and to be easily released from, a persons septum, as is the strap of the invention that is formed from a memory plastic material.

### SUMMARY OF THE INVENTION

The invention relates to devices for improving air flow through a person's nasal passages. The invention includes a pair of like short cylindrical shaped dilators that are of a diameter and material that is selected to flex to conform to and comfortable fit into each of the person's nasal cavities and includes a strap connected at its ends to the short cylinders lower edges, to span the septum. With the short cylinders fitting comfortably in the person's nasal passages, the strap, that is also formed from a material having a memory to be easily folded as to lightly pinch the persons septum and to hold that fold during use and is easily released should the person wish to remove the invention from their nasal cavities. The invention is preferably formed as a single unit from a flexible plastic material, such as a thermoplastic elastomer that will not react with the skin of the persons nostrils or septum and the preferred thermoplastic material is known as Santoprene that is manufactured by Exon Mobile. It should, however be understood, that the material from which the invention is formed can be any suitable material within the scope of this disclosure.

It is a principal object of the present invention to provide a device for improving an air flow through a person's nasal passages.

Another object of the present invention is to provide a device that is easily installed and comfortably worn in a person's nostrils during the day and/or night for improving an air flow through their nasal passages.

Still another object of the present invention is to provide a device for comfortably keeping a person's nasal passages open during the day and at night.

### BRIEF DESCRIPTION OF THE DRAWINGS

These and other objects and features of the present invention will become more apparent from the following description in which the invention is described in detail in conjunction with the accompanying drawings:

FIG. 1 is a side elevation perspective view of the invention in a clip-in nasal expander that includes a pair of like short cylinders that each connect to an end of a single strap;

FIG. 2 is a front elevation view of the clip-in nasal expander of FIG. 1 showing, with curved arrow A, the strap as being bent upon itself at its center,

FIG. 3 shows a front elevation view of the clip-in nasal expander held at the strap bend between a person's thumb and forefinger, with the short cylinders aligned for insertion into the person's nostrils; and

FIG. 4 shows a bottom plan view of the person's nose and nostrils of FIG. 3, showing the cylinders installed in the person's nostrils and with the strap clamped onto the person's septum.

### DETAILED DESCRIPTION

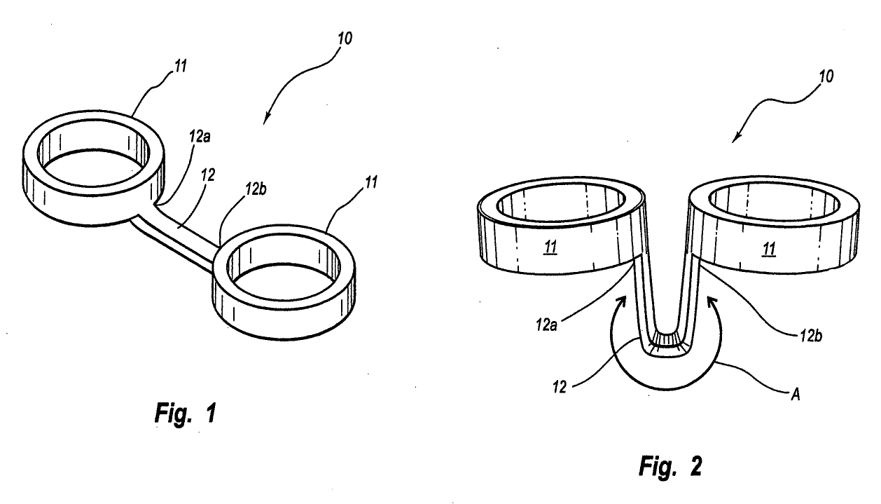

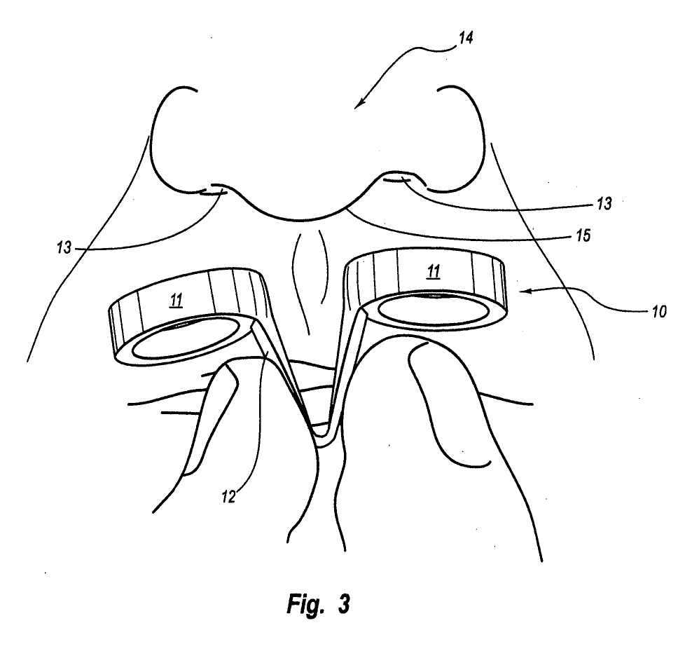

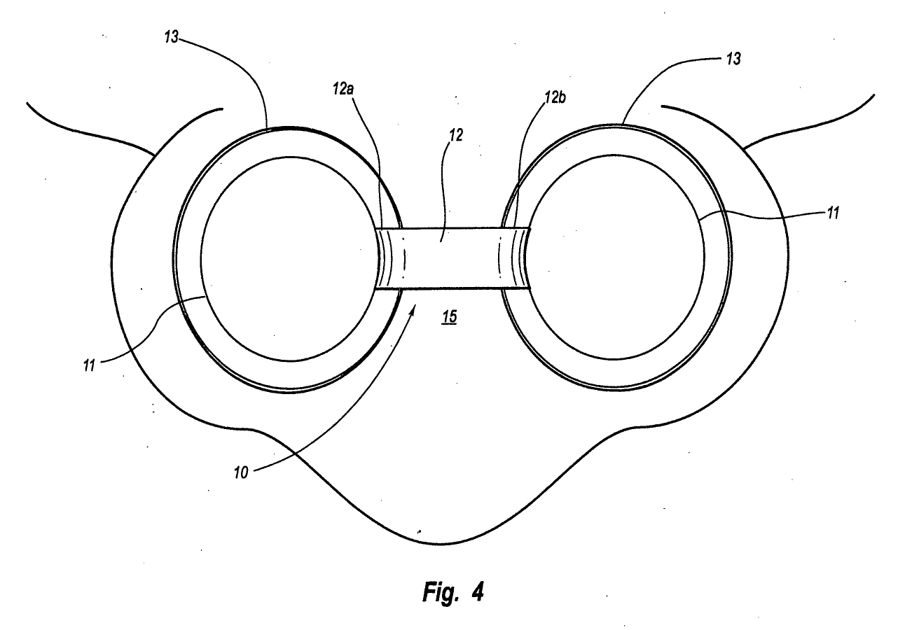

The invention, as is hereinafter described, is in a device for wear by a person during the day or night to provide, as shown in FIG. 3, for opening the nasal cavities 13 of nose 14. FIGS. 1 through 4 show a clip-in nostril expander 10 of the invention as including a pair of like size, identical, short cylindrical shaped dilators 11 that are each connected at 12a to ends of a strap 12. The dilators 11 are formed from a soft non-reactive plastic material, such as a thermoplastic elastomer known as Santoprene that is manufactured by Exon Moble, has been successfully used for the manufacture of the invention, In practice, the clip-in nostril expander 10 conforms to the person's nasal cavities 13, fitting snugly and comfortably therein. Which Santoprene provides for maintaining the dilators 11 in the person's nasal cavities, at their nostril ends, and provides to strap 12, as shown in FIGS. 2 and best in FIG. 3, with the ability to be bent, as shown by arrow A in FIG. 2, and to be fitted to, and clamp to the person's septum 15, as illustrated in FIGS. 3 and 4.

In practice, the strap 12 is bent at its center, as shown in FIG. 3, and holds that center bend to provide for a clamping action onto the person's septum 15, as shown in FIG. 4. After bending pressure is removed, the strap will retain the bend, as shown in FIG. 4. So arranged, the dilators 11 are comfortably fitted into the person's nostrils and will conform to and retain the shape of each nostril. With which nostril shape retention and the retention of the bend in the strap 12, the individual dialators are firmly and comfortably maintained in the person's nostrils. To provide which memory function to both the dialators 11 and strap 12, the clip-in nostril expander 10 is manufactured as a single unit from a memory plastic material that is easily formed or bent to desired shape. A material as has been used to manufacture the invention is a thermoplastic elastomer material known as Santoprene manufactured by Exon Mobile that will not react with the skin of a person's nostrils or septum and has the memory characteristics required to provide for a conformity of the dialators 11 with the persons nostrils and the strap 12 with the required clamping ability.

With the positioning of the clip-in nostril expander dilators 11 in a person's nostrils breathing takes place through the dilators 11, greatly lessening the exertion that was formerly necessary to pass air in and out of the nose, without a necessity of undergoing a surgical procedure as has often been prescribed for breathing problems. In practice, problems with snoring have often been cured with a first overnight installation of the device, and persons have uniformly reported improvements in breathing with little or no discomfort.

Hereinabove has been set out a description of a preferred embodiment of the clip-in nostril expander of the invention. It should however, be understood that the present invention can be varied within the scope of this disclosure without departing from the subject matter coming within the scope of the following claims, and a reasonable equivalency thereof, which claims I regard as my invention.

I claim:

1. A clip-in nostril expander comprising, a pair of separate like dilators each formed from an elastomeric material having a flat cylindrical shape with a diameter to comfortably fit in each of a person's nostrils and a strap that is connected at its ends to each of said dialators and is formed from an elastomeric material having a capability to maintain a bend therein, providing for clamping to the skin of a person's septum between their nostrils, and where said dilators and connecting strap are formed as a single unit from a thermoplastic elastomer that is a memory plastic that is capable of maintaining a shape as it is squeezed or bent into.

2. The clip-in nostril expander as recited in claim 1, where the dilators and strap are formed as a single unit from a thermoplastic Santoprene.
